(19) United States
(12) Patent Application Publication
(10) Pub. No.: US 2025/0135074 A1
(43) Pub. Date: May 1, 2025

(54) ZnO-REINFORCED Mg-Zr MATRIX BIOCOMPOSITES AND METHODS OF PREPARATION THEREOF

(71) Applicant: KING FAHD UNIVERSITY OF PETROLEUM AND MINERALS, Dhahran (SA)

(72) Inventors: Mohamed Abdrabou HUSSEIN, Dhahran (SA); Mohammed Abdul AZEEM, Dhahran (SA); Arumugam Madhan KUMAR, Dhahran (SA)

(73) Assignee: KING FAHD UNIVERSITY OF PETROLEUM AND MINERALS, Dhahran (SA)

(21) Appl. No.: 18/494,837

(22) Filed: Oct. 26, 2023

(51) Int. Cl. A61L 31/12; A61L 31/02; A61L 31/14; B22F 3/16; C22C 1/04; C22C 1/059; C22C 23/00

(52) U.S. Cl. CPC A61L 31/124; A61L 31/026; A61L 31/146; B22F 3/16; C22C 1/0408; C22C 1/059; C22C 23/00; B22F 2201/10; B22F 2301/058; B22F 2301/205; B22F 2302/25; B22F 2304/10; B22F 2998/10; B22F 2999/00

(57) ABSTRACT

A method of making a composite, including mixing ZnO nanoparticles (NPs), Mg particles, and Zr particles under an inert atmosphere to form a powder mixture, compacting the powder mixture at a pressure of 500-600 MPa for at least 1 minute to form a compacted mixture, and sintering the compacted mixture at a temperature of 400-500° C. for at least 1 hour to form the composite. The composite includes 1-10 wt. % of the ZnO NPs and 0.1-5 wt. % of the Zr particles, based on a total weight of the composite, the Zr particles and the ZnO NPs are homogeneously dispersed in a matrix of the Mg particles in the composite, the Mg particles have an average grain size of 5-10 μm in the composite, and the Zr particles and the ZnO NPs separately form aggregates at grain boundaries of the Mg particles in the composite.

### BACKGROUND

### Technical Field

The present disclosure is directed to a composite for bioimplant applications, and more particularly to a method for fabricating ZnO-reinforced Mg—Zr matrix composites for bioimplant applications.

### Description of Related Art

The “background” description provided herein is to present the context of the disclosure generally. Work of the presently named inventors, to the extent it is described in this background section, as well as aspects of the description that may not otherwise qualify as prior art at the time of filing, are neither expressly nor impliedly admitted as prior art against the present invention.

Magnesium has a low elastic modulus and a high degradation rate compared to other prominent biomaterials such as stainless-steel and titanium, making it a promising choice as a short-term implant material in the healing stage, thereby avoiding removal surgery. However, the degradation rate through corrosion in physiological conditions should be controlled to avoid implant failure, inflammatory reactions, and microbial activity. Moreover, the physiochemical and mechanical properties of Mg-based implants should be favorable to human physiology. These considerations have led to various studies and development of magnesium-based composites and alloys.

The biodegradability of Mg is commonly controlled by alloying it with various metals and rare earth elements. However, Mg alloys such as AE21, AZ91, AJ62, etc., have shown detrimental results due to galvanic coupling with alloying elements and neurotoxic effects. Mg alloys have also been tuned with surface coatings, which can have a complicated biodegradation period. Hence, the choice and amount of alloying element in a Mg composite is selected to control the degradation rate and to avoid toxicity. Mg alloyed with non-toxic or nutrient-based elements such as Zn, Ca, Ag, Sr, Zr, and Mn have shown improvement in biodegradability. Similarly, Mg composites reinforced with bio-ceramics have been developed to cater to degradation rate and mechanical behavior. Namely, composites containing TiO₂, Al₂O₃, ZrO₂, and TiB₂ particles have enhanced corrosion resistance and superior mechanical properties over Mg alloys. Mg composites involving reinforcements similar to the composition of human bone, such as CPP, HAP, and β-TCP, have shown enhanced biocompatibility and osteoconductive properties, however, their inferior mechanical behavior may limit their application.

Zinc oxide (ZnO) has been used in the medical industry due to its benefits, such as biodegradability, biocompatibility, low toxicity, antibacterial, wound healing, anti-inflammation, and tissue integration ability. Further, ZnO nanoparticles (NPs) have improved properties such as high specific surface area, high bioactivity, physiochemical properties, in vitro biodegradability, and cytocompatibility in bone tissue engineering applications. However, their mechanical behavior is inferior to metallic biomaterials. Mg reinforced with ZnO NPs have improved strength and wear characteristics of Mg composites at higher ZnO loadings, however, the effect of a low concentration of ZnO (below the solubility limit of Zn in Mg) on the biodegradability of Mg is unknown.

A factor in corrosion protection or biodegradation in Mg composites is grain structure refinement, which enables quick passivation of an implant surface, and other metallurgical factors such as secondary phases, texture, or crystallography. Hence various grain refining elements, post-processing methods, and reinforcement additions have been under investigation. Zirconium (Zr) is one the most commonly used alloying element for Mg and Ti, due to its biocompatibility, corrosion resistance, and antimicrobial properties. Studies incorporating Zr in the Mg matrix have improved corrosion resistance due to grain refinement. Tribological properties are another factor for the performance of implant materials. High wear resistance is desired to prevent body inflammation while a considerable value of coefficient of friction aids in preventing the loosening of implants.

Although several composites have been developed in the past for bioimplant applications, there still exists a need to fabricate composites with improved properties. It is one object of the present disclosure to provide a composite with improved grain refinement and tribological properties.

### SUMMARY

In an exemplary embodiment, a method of making a composite is described. The method includes mixing zinc oxide (ZnO) nanoparticles (NPs), magnesium (Mg) particles, and zirconium (Zr) particles under an inert atmosphere to form a powder mixture; compacting the powder mixture at a pressure of 500-600 Megapascal (MPa) for at least 1 minute (min) to form a compacted mixture; and sintering the compacted mixture at a temperature of 400-500 degrees Celsius (° C.) for at least 1 hour (h) to form the composite, wherein the composite includes 1-10 wt. % of the ZnO NPs and 0.1-5 wt. % of the Zr particles, based on a total weight of the composite. The Zr particles and the ZnO NPs are homogeneously dispersed in a matrix of the Mg particles in the composite, wherein the Mg particles have an average grain size of 5-10 micrometers (μm) in the composite, and wherein the Zr particles and the ZnO NPs separately form aggregates at grain boundaries of the Mg particles in the composite.

In some embodiments, the mixing the ZnO NPs have an average size of 80-200 nanometers (nm).

In some embodiments, the mixing the Mg particles have an average particle size of 30-60 μm.

In some embodiments, the mixing the Zr particles have an average particle size of 30-60 μm.

In some embodiments, composite includes Mg, ZnO, and MgO crystal phases.

In some embodiments, composite does not include a MgZn crystal phase.

In some embodiments, Mg particles include α-Mg in the composite.

In some embodiments, aggregates of the ZnO NPs are from 0.5-10 μm in size.

In some embodiments, aggregates of the Zr particles are from 0.5-10 μm in size.

In some embodiments, composite has a density of 1.7-1.8 gram per cubic centimeter (g/cm³).

In some embodiments, composite has less than 2% porosity.

In some embodiments, composite has a Vickers hardness of 70-80 Vickers Pyramid Number (HV).

In some embodiments, composite has a specific wear rate of 0.001 to 0.002 cubic millimeters per Newton per meter (mm³/Nm).

In some embodiments, composite has a corrosion rate of less than millimeters/year (0.1 mm/yr) in a human body fluid solution.

In some embodiments, a composite is made by mixing ZnO NPs, Mg particles, and Zr particles under an inert atmosphere to form a powder mixture; compacting the powder mixture at a pressure of 500-600 MPa for at least 1 min to form a compacted mixture; and sintering the compacted mixture at a temperature of 400-500° C. for at least 1 h to form the composite.

In some embodiments, an implant is described as containing the composite.

In some embodiments, the implant has a corrosion rate of less than 0.1 mm/yr in a human body fluid solution.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a flowchart depicting a method of making a ZnO-reinforced Mg—Zr composite, according to certain embodiments;

FIG. 2A shows a scanning electron microscopic (SEM) image of a Mg—ZnO composite powder before sintering, according to certain embodiments;

FIGS. 2B-2D show elemental maps of mixed Mg—ZnO composite powder, depicting magnesium (Mg), oxygen (O), and zinc (Zn) respectively, before sintering, according to certain embodiments;

FIG. 3A shows a SEM image of the Mg—Zr—ZnO composite powder before sintering, according to certain embodiments;

FIGS. 3B-3E show elemental maps of mixed Mg—Zr—ZnO composite powder, depicting magnesium (Mg), zirconium (Zr), zinc (Zn), and oxygen (O), respectively, before sintering, according to certain embodiments;

FIG. 4A shows a digital micrograph of pristine Mg at 20 μm scale, according to certain embodiments;

FIG. 4B shows a digital micrograph of the Mg—ZnO composite at 20 μm scale, according to certain embodiments;

FIG. 4C shows a digital micrograph of the Mg—Zr—ZnO composite at 20 μm scale, according to certain embodiments;

FIG. 5 shows X-ray diffraction (XRD) patterns of (A) pure Mg, (B) Mg—ZnO composite, and (C) Mg—Zr—ZnO composite after sintering, according to certain embodiments;

FIGS. 6A-6B show Field Emission Scanning Electron Microscopy (FESEM) backscattered micrographs of sintered Mg—ZnO at different magnifications, according to certain embodiments;

FIGS. 6C-6D show FESEM backscattered micrographs of the sintered Mg—Zr—ZnO composite at different magnifications, according to certain embodiments;

FIG. 7A shows a high magnification SEM micrograph for the sintered Mg—ZnO composite, according to certain embodiments;

FIGS. 7B-7D show elemental maps of the sintered Mg—ZnO composite depicting Mg, Zn, and O, respectively, according to certain embodiments;

FIG. 7E shows a high magnification SEM micrograph for the sintered Mg—Zr—ZnO composite, according to certain embodiments;

FIGS. 7F-7I show elemental maps of the sintered Mg—Zr—ZnO composite, depicting Mg, Zr, Zn, and O, respectively, according to certain embodiments;

FIG. 8A shows a coefficient of friction (COF) curve for all samples with respect to time during the sliding wear test, according to certain embodiments;

FIG. 8B shows a plot for specific wear rate measured for all samples at constant wear testing conditions, according to certain embodiments;

FIGS. 8C-8E show the 3D optical profilometer images of wear track in tested pure Mg, Mg—ZnO, and Mg—Zr—ZnO composite samples, respectively, according to certain embodiments;

FIG. 9A shows a SEM image of worn surface (inside wear track core) of the pure Mg, according to certain embodiments;

FIG. 9B shows the corresponding EDS spectrum, of FIG. 9A, according to certain embodiments;

FIG. 9C shows a SEM image of worn surface (inside wear track core) of the Mg—ZnO composite, according to certain embodiments;

FIG. 9D shows the corresponding EDS spectrum of FIG. 9C, according to certain embodiments;

FIG. 9E shows a SEM image of worn surface (inside wear track core) of the Mg—Zr—ZnO composite, according to certain embodiments,

FIG. 9F shows the corresponding EDS spectrum of FIG. 9E, according to certain embodiments;

FIG. 10 shows open-circuit potential (OCP) curves of prepared Mg composites samples in simulated body fluid (SBF) medium, according to certain embodiments;

FIG. 11A shows linear polarization resistant (LPR) curves of the pure Mg in SBF medium, according to certain embodiments;

FIG. 11B shows LPR curves of the Mg—ZnO composite in the SBF medium, according to certain embodiments;

FIG. 11C shows LPR curves of the Mg—Zr—ZnO composite in the SBF medium, according to certain embodiments;

FIG. 12 shows potentiodynamic polarization (PDP) curves for prepared composites in SBF medium, according to certain embodiments;

FIG. 13A shows Nyquist plots of the prepared composites in SBF medium, according to certain embodiments;

FIG. 13B shows Bode plots of the prepared composites in SBF medium, according to certain embodiments;

FIG. 14A shows electrochemical impedance spectroscopic (EIS) circuit model for the Mg composite in SBF medium, according to certain embodiments; and

FIG. 14B shows EIS circuit models for Mg—ZnO and Mg—Zr—ZnO composites in SBF medium, according to certain embodiments.

### DETAILED DESCRIPTION

When describing the present disclosure, the terms used are to be construed in accordance with the following definitions, unless a context dictates otherwise.

Embodiments of the present invention will now be described more fully hereinafter with reference to the accompanying drawings wherever applicable, in that some, but not all embodiments of the disclosure are shown.

In the drawings, reference numerals designate identical or corresponding parts throughout the several views. Further, as used herein, the words “a,” “an,” and the like generally carry a meaning of “one or more,” unless stated otherwise.

Where a numerical limit or range is stated herein, the endpoints are included. Also, all values and subranges within a numerical limit or range are specifically included as if explicitly written out.

Furthermore, the terms “approximately,” “approximate,” “about,” and similar terms generally refer to ranges that include the identified value within a margin of 20%, 10%, or preferably 5%, and any values therebetween.

As used herein, the term “composite material” refers to an amalgamation of at least two materials with distinct physical and chemical properties.

As used herein, the term “nanoparticles” or “NPs” refers to particles having a particle size of 1 nanometer (nm) to 1,000 nm within the scope of the present invention.

As used herein, the term “porosity” refers to a measure of the void or vacant spaces within a material.

As used herein, the term “particle size” and “pore size” may be thought of as the lengths or longest dimensions of a particle and of a pore opening, respectively.

As used herein, the term “grain” also known as crystallite refers to a tiny or even microscopic crystal which is formed during the cooling of many materials.

As used herein, the term “grain boundary” refers to an interface among two grains (crystallites), in a polycrystalline material.

As used herein, “density” of a material refers to its mass per unit volume. Density is the measure of how much mass is contained in a given volume. It is an intrinsic property of an object.

As used herein, the term “powder metallurgy” refers to a process where compacted metal powders are heated to just below melting point to form metal parts.

As used herein, the term “milling” refers to the process of grinding or pulverizing larger materials to achieve a specific level of fineness.

As used herein, the term “compacting” refers to a compression molding process that utilizes a press (usually hydraulic) to construct complex shapes from composite, metallic, ceramic, and other powder compounds.

As used herein, the term “sintering” refers to a process of compacting and forming material into a solid mass, using pressure and heat without melting it to a liquid state.

As used herein, the term “wear” refers to the gradual removal or deformation of material at solid surfaces caused by mechanical or chemical factors.

As used herein, the term “corrosion” refers to the conversion of materials, for instance, metals into more stable forms. There are two main types of corrosion, general or uniform attack corrosion, and galvanic corrosion. Typical or uniform corrosion happens, for instance, when the iron is in a humid environment, creating iron oxide and corroding.

As used herein, the term “biomaterial” is any synthetic or natural substance that can be utilized in contact with biological systems in order to evaluate, treat, augment, or replace any tissue, organ, or function of the body.

As used herein, the term “bioimplant” refers to any biosynthetic implantable prosthesis that replaces, supports or enhances a failing structure in the body of a subject.

As used herein, the term “Vickers Hardness” is a measure of the hardness of a material, assessed from the size of an impression produced under load using a pyramid-shaped diamond indenter. The unit of hardness given by the Vickers Hardness test is called the Vickers Pyramid Number (HV) or Diamond Pyramid Hardness (DPH).

As used herein, the term “microhardness testing” is a method for measuring the surface of small parts or areas. It can also be used for measuring individual microstructures or the depth of case hardening by making a series of indentations and creating a profile of the change in hardness.

As used herein, the term “specific wear rate” refers to the volume of material worn away per unit of sliding distance and per unit of load. It is evaluated as the wear volume divided by the product of the normal load and the sliding distance with a unit of cubic millimeters per Newton per meter (mm³/Nm).

As used herein, the term “coefficient of friction (COF)” refers to the ratio of the frictional force to the normal force. It is dependent on the surface roughness of the contacting materials.

As used herein, the term “open-circuit potential (OCP)” refers to the difference of electrical potential between two terminals of a device when the circuit is broken or open or there is not any external load connected.

As used herein, the term “simulated body fluid (SBF)” refers to a solution with an ion concentration similar to that of human blood plasma, maintained at a pH and temperature that are physiologically mild. In a preferred embodiment, the SPF has a sodium ion concentration of 142 mM, a potassium ion concentration of 5 mM, a magnesium ion concentration of 1.5 mM, a calcium ion concentration of 2.5 mM, a chloride ion concentration of 148 mM, a bicarbonate ion concentration of 4.2 mM, a hydrogen phosphate ion concentration of 1 mM, and a sulfate ion concentration of 0.5 mM, in a tris(hydroxymethyl)aminomethane (tris) buffer solution.

A weight percent of a component, unless specifically stated to the contrary, is based on the total weight of the formulation or composition in which the component is included. For example, if a particular element or component in a composition or article is said to have 5 wt. %, it is understood that this percentage is in relation to a total compositional percentage of 100%.

The present disclosure is intended to include all hydration states of a given compound or formula, unless otherwise noted or when heating a material. The present disclosure is intended to include all isotopes of a given compound or formula, unless otherwise noted.

Aspects of the present disclosure are directed towards a method of making and the resultant mechanical, corrosion, and wear behavior of ZnO NPs reinforced Mg—Zr composites for bioimplant applications. The processed Mg—Zr—ZnO composite has improved microhardness, corrosion, and wear characteristics, making it a potential bioimplant material.

In some embodiments, the composite includes Mg, Zr, and ZnO. In some embodiments, the composite may further include or the Mg can be at least partially replaced with calcium, strontium, or beryllium. In some embodiments, the composite may further include or the Zr can be replaced with niobium, molybdenum, technetium, rhodium, ruthenium, palladium, or silver. In some embodiments, the composite may further include or the ZnO can be at least partially replaced with TiO₂, Al₂O₃, ZrO₂, and TiB₂. In an embodiment, the additional element and/or oxide is included in an amount of 0.1-10 wt. %, preferably 2-9 wt. %, preferably 3-8 wt. %, preferably 4-7 wt. %, or preferably 5-6 wt. %, based on a total weight of the composite. In a preferred embodiment, taking into account cost and biocompatibility, the composite consists of Mg, Zr and ZnO.

In some embodiments, the Mg is used as a matrix in the composite and is the bulk of the composite. In some embodiments, the composite includes at least 50 wt. % of the Mg, preferably 55 wt. %, 60 wt. %, 65 wt. %, 70 wt. %, 75 wt. %, 80 wt. %, 85 wt. %, 90 wt. %, 95 wt. %, or about 97 wt. %. In some embodiments, the composite includes 1-10 wt. % of the ZnO, preferably 2-9 wt. %, preferably 3-8 wt. %, preferably 4-7 wt. %, preferably 5-6 wt. % of the ZnO and 0.1-5 wt. % of the Zr particles, preferably 0.5-4.5 wt. %, preferably 1-4 wt. %, preferably 1.5-3.5 wt. %, preferably 2-3 wt. %, based on a total weight of the composite. In a specific embodiment, the composite includes 2.5 wt. % of the ZnO, and 1 wt. % of the Zr particles, based on a total weight of the composite.

In some embodiments, the Mg particles may include alpha (α), beta (β), and/or gamma (γ) phases. In a specific embodiment, the Mg particles include α-Mg in the composite. In some embodiments, Mg particles have an average grain size of 5-10 micrometers (μm), preferably 6-9 μm, and preferably 7-8 μm in the composite. In a specific embodiment, Mg particles have an average grain size of 8.7 μm in the Mg—Zr—ZnO composite. In some embodiments, the composite does not include a MgZn crystal phase, in other words there are not metal-metal formations in the composite. In some embodiments, the composite includes Mg, ZnO, and MgO crystal phases.

In some embodiments, the ZnO is in the form of ZnO nanoparticles (NPs). The NPs may exist in various morphological shapes, such as nanowires, nanocrystals, nanorectangles, nanotriangles, nanopentagons, nanohexagons, nanoprisms, nanodisks, nanocubes, nanoribbons, nanoblocks, nanobeads, nanotoroids, nanodiscs, nanobarrels, nanogranules, nanowhiskers, nanoflakes, nanofoils, nanopowders, nanoboxes, nanostars, tetrapods, nanobelts, nano-urchins, nanofloweres, etc., and mixtures thereof. In a preferred embodiment, the ZnO nanoparticles have a spherical shape with an average diameter of 10-200 nm, preferably 30-180 nm, 50-160 nm, 70-140 nm, 90-120 nm, or about 100 nm, in the composite.

In some embodiments, the Zr particles and the ZnO NPs are homogeneously dispersed in a matrix of the Mg particles in the composite. In some embodiments, the Zr particles and the ZnO NPs separately form aggregates at grain boundaries of the Mg particles in the composite. In other words, the ZnO NPs aggregate at the edge of a Mg grain at a junction adjacent to another Mg grain and separately the Zr particles aggregate at the edge of a Mg grain at a junction adjacent to another Mg grain.

In some embodiments, the aggregates of the ZnO NPs are from 0.5-10 μm in size (largest dimension), preferably 1.0-9.5 μm, preferably 1.5-9.0 μm, preferably 2.0-8.5 μm, preferably 2.5-8.0 μm, preferably 3.0-7.5 μm, preferably 3.5-7.0 μm, preferably 4.0-6.5 μm, preferably 4.5-6.0 μm, preferably 5.0-5.5 μm in size. In some embodiments, the aggregates of the Zr particles are from 0.5-10 μm in size, preferably 1.0-9.5 μm, preferably 1.5-9.0 μm, preferably 2.0-8.5 μm, preferably 2.5-8.0 μm, preferably 3.0-7.5 μm, preferably 3.5-7.0 μm, preferably 4.0-6.5 μm, preferably 4.5-6.0 μm, preferably 5.0-5.5 μm in size. In a preferred embodiment, the Zr aggregates are larger than the ZnO aggregates.

In some embodiments, the composite has a density of 1.7-1.8 g/cm³, preferably 1.71-1.79 g/cm³, preferably 1.72-1.78 g/cm³, preferably 1.73-1.77 g/cm³, preferably 1.74-1.77 g/cm³, and preferably 1.75-1.76 g/cm³. In a specific embodiment, the composite has a density of 1.762 g/cm³. In some embodiments, the composite has less than 2% porosity, preferably 1.5%, preferably 1%, preferably 0.5%, preferably 0.1%. In a specific embodiment, the composite has a porosity of 1.13±0.18%.

In some embodiments, the composite has a Vickers hardness of 70-80 HV, preferably 71-79 HV, preferably 72-78 HV, preferably 73-77 HV, and preferably 74-76 HV. In a specific embodiment, the composite has a Vickers hardness of 76.5±3 HV. In a specific embodiment, the Vickers hardness was enhanced by 50% for Mg—Zr—ZnO composite in comparison to pure Mg prepared by the same method.

In some embodiments, the composite has a specific wear rate of 0.001-0.002 mm³/Nm, preferably 0.0011-0.0019, preferably 0.0012-0.0018, preferably 0.0013-0.0017, and preferably 0.0014-0.0016 mm³/Nm. In a specific embodiment, the composite has a specific wear rate of 0.00154 mm³/Nm. In a specific embodiment, the wear rate was reduced by 35% for Mg—Zr—ZnO composite in comparison to pure Mg prepared by the same method.

In some embodiments, the composite has a corrosion rate of less than 0.1 mm/yr, preferably 0.09, preferably 0.08, preferably 0.07, preferably 0.06, preferably 0.05, preferably 0.04, preferably 0.03, preferably 0.02, preferably 0.01 mm/yr in a human body fluid solution. In a specific embodiment, the composite has a corrosion rate of 0.056 mm/yr in a human body fluid solution. The corrosion rate of the composite is lower than that of pure Mg prepared by the same method.

While not wishing to be bound to a single theory, it is thought that the incorporation of both the Zr and the ZnO into the nanocomposite results in grain refinement which provides synergetic results. The Mg grains are refined from thick and large grain boundaries to finer grains and thin grain boundaries in the composite. Further, aggregation of the Zr and ZnO at the grain boundaries increase the density and decrease the porosity, thereby resulting in increased hardness and resistance to wear and corrosion. These characteristics improve the potential for use as a biomedical implant, particularly in avoiding the self-loosening of implants, enhanced durability and extended lifespan.

The composite of the present disclosure may be used in a bioimplant. In a preferred embodiment, the implant is an orthopedic implant. Examples of orthopedic implants include but are not limited to pins, rods, screws, and plates used to anchor fractured bones while they heal. In a preferred embodiment, the implant is in place long enough for the bone to heal but degrades over time and does not require removal surgery. In some embodiments, the implant containing the composite has a corrosion rate of less than 0.1 mm/yr, preferably 0.09, preferably 0.08, preferably 0.07, preferably 0.06, preferably 0.05, preferably 0.04, preferably 0.03, preferably 0.02, preferably 0.01 mm/yr in a human body fluid solution. In a specific embodiment, the composite has a corrosion rate of 0.056 mm/yr in a human body fluid solution.

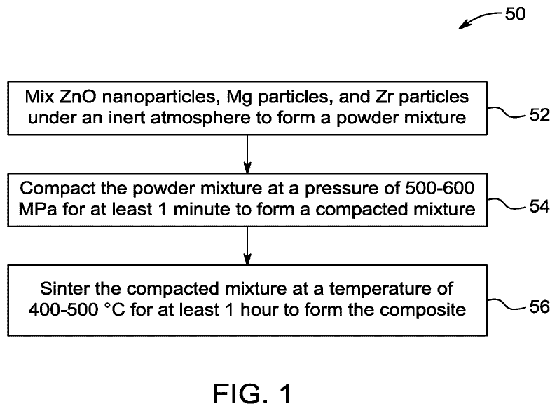

FIG. 1 illustrates a flow chart of a method 50 of making a composite. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes mixing ZnO NPs, Mg particles, and Zr particles under an inert atmosphere to form a powder mixture. The mixing is usually carried out manually, with the help of a stirrer, or using milling process. The inert atmosphere can be provided nitrogen, helium, and argon. In a specific embodiment, the inert atmosphere is provided by argon gas.

In some embodiments, the ZnO nanoparticles have an average size of 80-200 nm, preferably 85-195 nm, preferably 90-190 nm, preferably 95-185 nm, preferably 100-180 nm, preferably 105-175 nm, preferably 110-170 nm, preferably 115-165 nm, preferably 120-160 nm, preferably 125-155 nm, preferably 130-150 nm, and preferably 135-145 nm. In some embodiments, in the mixing the Mg particles have an average size of 30-60 μm, preferably 35-55 μm, and preferably 40-50 μm. In a specific embodiment, the Mg particles have an average size of 45 μm. In some embodiments, in the mixing the Zr particles have an average size of 30-60 μm, preferably 35-55 μm, and preferably 40-50 μm. In an embodiment, the Mg and the Zr are in the form of powders. The powders include the pure metal and do not include oxides thereof.

At step 54, the method 50 includes compacting the powder mixture at a pressure of 500-600 Megapascal (MPa), preferably 505-595 MPa, preferably 510-590 MPa, preferably 515-585 MPa, preferably 520-580 MPa, preferably 525-575 MPa, preferably 530-570 MPa, preferably 535-565 MPa, preferably 540-560 MPa, and preferably 545-555 MPa for at least 1 minute (min), preferably 2 min, preferably 3 min, preferably 4 min, and preferably 5 min to form a compacted mixture. The compacting is for at most 1 hour, preferably 50 mins, 40 mins, 30 mins, 20 mins, or 10 mins. In a specific embodiment, the powder mixture is compacted at 550 MPa for 5 min. In one embodiment, a uniaxial 40-ton hydraulic press was used for compaction.

At step 56, the method 50 includes sintering the compacted mixture at a temperature of 400-500° C., preferably 405-495° C., preferably 410-490° C., preferably 415-485° C., preferably 420-480° C., preferably 425-475° C., preferably 430-470° C., preferably 435-465° C., preferably 440-460° C., and preferably 445-455° C. for at least 1 hour (h), preferably 1.5 h, and preferably 2 h to form the composite. The sintering is for at most 10 h, preferably 9 h, 8 h, 7 h, 6 h, 5 h, 4 h, or 3 h. In a specific embodiment, the compacted mixture was sintered in a tube furnace at 450° C. for 2 h.

### EXAMPLES

The following examples demonstrate a method for the fabrication of ZnO-reinforced Mg and Mg—Zr matrix composites for bioimplant applications as described herein. The examples are provided solely for illustration and are not to be construed as limitations of the present disclosure, as many variations thereof are possible without departing from the spirit and scope of the present disclosure.

### Example 1: Materials

Magnesium (Mg) powder (99.8 percent purity with an average particle size of 45 m) as a matrix was used. Zirconium (Zr) powder (−325 mesh size and 99.8% purity) and zinc oxide (ZnO) nanoparticles (NPs) (size 80-200 nm, from US research nanomaterials, Inc.) were used as reinforcements. Mg and Zr powders were obtained from Alfa Aesar and used without any modification.

### Example 2: Synthesis of Mg-Based Composites

Initially, to synthesize an Mg-based composite, elemental powders were blended as Mg with 2.5 wt. % ZnO (Mg—ZnO) and Mg with 1 wt. % Zr and 2.5 wt. % ZnO (Mg—Zr—ZnO) and mixed thoroughly using a planetary Micro Mill Pulverisette 7 (FRITSCH, Germany). The powders were mixed for 2 hours at a rotational speed of 200 rpm under an argon atmosphere inside WC vials. After mixing, the composite powders were loaded in tool steel die with a bore diameter of 20 mm for compaction. A uniaxial 40-ton hydraulic press (Carver, USA) was used to compact at 550 MPa pressure with a holding time of 5 min. The green compacts were sintered in a tube furnace (GSL-1700X, MTI) at 450° C. for 2 hrs. at a heating rate of 10° C./min under an argon atmosphere. A Mg composite sample was also prepared as a control under the same conditions (labeled as pure Mg).

### Example 3: Characterization Techniques

The elemental powders after mixing were characterized using scanning electron microscopic (SEM) equipped with elemental X-ray spectroscopic (EDS) mapping. The microstructural analysis of the sintered composite were investigated using Field Emission Scanning Electron Microscopy (FESEM) (FEI Quanta 250 FEG, USA). The composition and elemental distribution in synthesized samples were evaluated by EDS. The samples for the metallographic study were prepared by grinding at a slow speed on a series of SiC abrasives (grit size from 240-1200). This was followed by polishing using alumina (0.05 micrometers (μm) powder) suspension. The samples were immediately etched using acetic picral and analyzed under a digital microscope (DSX series, Olympus). The XRD analysis was done on a Bruker-AXS D8 (manufactured by Brukker, Billerica, Massachusetts, United States) diffractometer using Cu Kα (λ=1.5418 Å) radiation scanned with a step size of 0.02°.

### Example 4: Density and Microhardness Measurement Techniques

The density of sintered samples was measured using high precision electronic densimeter (MDS-300, Alfa Mirage, Japan). The weight of the sample in air and subsequently in distilled water was measured to calculate density based on the Archimedes principle. The experimental densities of sintered samples obtained from the above procedure are recorded and compared to the theoretical density of the composite calculated according to the rule of mixtures. The microhardness of sintered samples was determined using a Microhardness tester (NG-1000, NextGen, Canada). Vickers hardness (HV) was measured at 50 gf load with a dwell time of 10 sec. The hardness value represents the average of 15 readings taken in a straight line on a flat, polished surface.

### Example 5: Wear Test

The samples were subjected to wear testing using a Bruker UMT-3 tribometer with a ball-on-disk configuration as per ASTM test standard G99-05. The sample was secured to a rotating turntable and a 6.3 mm diameter ball made of 440C stainless steel with a hardness of 62HRC was used as a counter-face. Wear tests were conducted at a load of 10 N, and at a constant sliding speed of 0.1 m/s (239 rpm) for 200 meters under dry conditions and 55±5% relative humidity at room temperature. The wear track profiles were analyzed using the GTK-A optical profilometer from Bruker to determine the wear track depth and cross-sectional area. Additionally, the wear volume was determined by multiplying the cross-sectional area with the average circumference of the entire wear track, and this value was used to estimate the specific wear rate.

### Example 6: Corrosion investigation

In vitro corrosion behavior of prepared Mg composite samples in simulated body fluid (SBF) was studied using the Gamry electrochemical instrument (Reference 6000, USA) through three electrodes set up in which Mg composite (exposed area of 1.76 cm²), graphite rod, saturated calomel electrodes respectively performing as working, counter and reference electrodes. Before initiating any electrochemical experiments, the open circuit potential (OCP) was scanned for about 30 minutes (min) to attain electrochemical stability in the SBF medium. Linear polarization resistant (LPR) tests were performed by selecting the potential of 25 mV with the sweeping rate of 0.125 cm². Electrochemical impedance spectroscopic (EIS) curves were obtained by applying the frequency range of 1 kHz to 1 mHz through 10-mV perturbation signal. Potentiodynamic polarization (PDP) measurements were done by selecting the applied potential of +250 mV vs OCP using a sweeping rate of 1 mV/s. All the obtained electrochemical findings were further quantitatively investigated through the Echem in-building analysis, and the reported data were replicated to confirm the reproducibility of the results.

### Example 7: Characterization

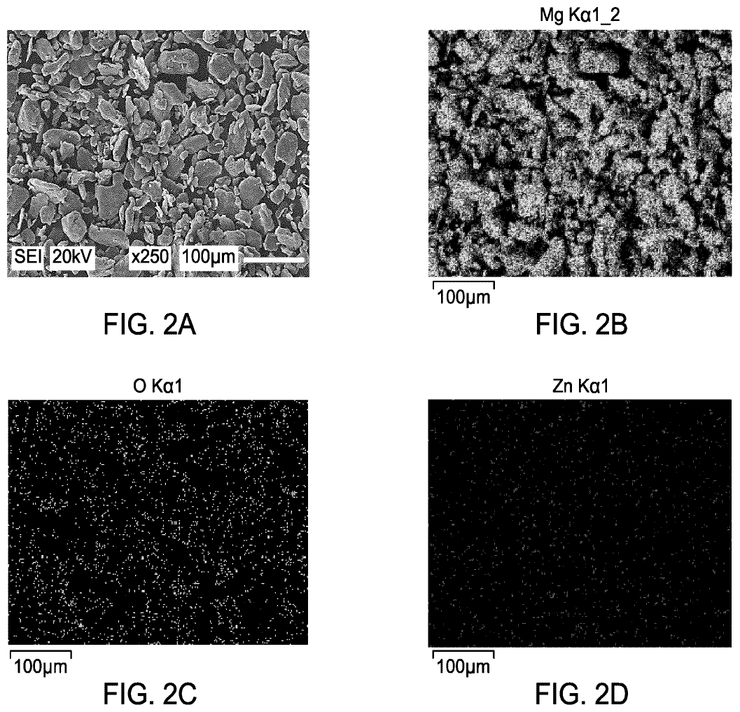

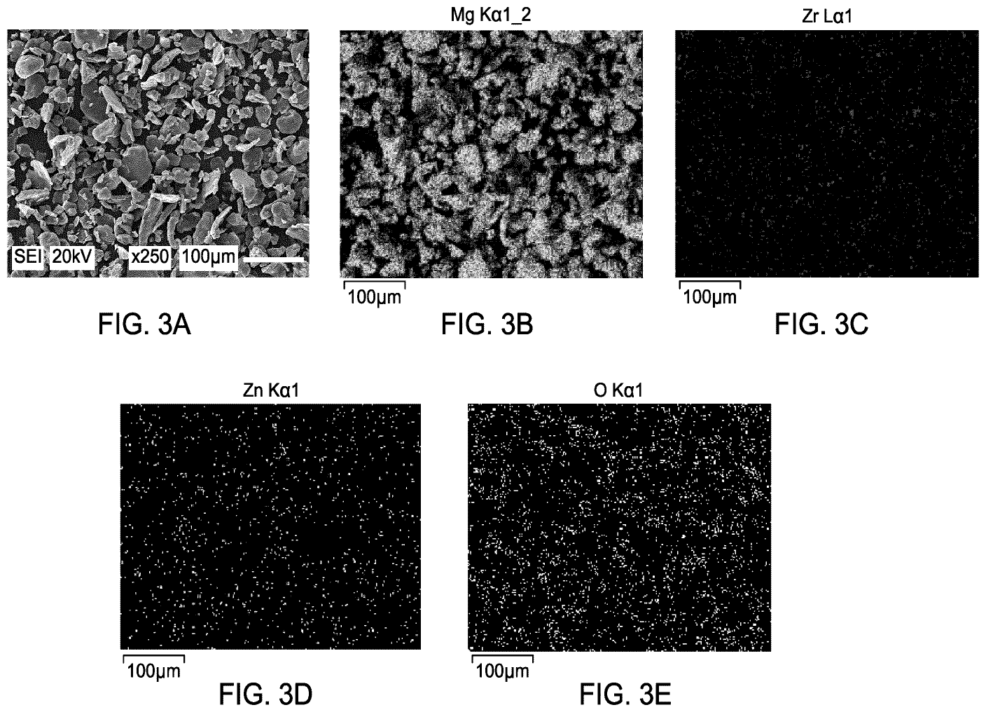

Before sintering, the blended powders of Mg, Zr, and ZnO particles were analyzed under SEM imaging/mapping to evaluate and confirm the uniform distribution of elements. FIG. 2A shows a scanning electron microscopic (SEM) image and FIGS. 2B-2D show elemental maps of mixed Mg—ZnO composite powder, depicting magnesium (Mg), oxygen (O), and zinc (Zn) respectively, before sintering. Whereas FIG. 3A shows a SEM image and FIGS. 3B-3E show elemental maps of the Mg—Zr—ZnO composite powder, depicting magnesium (Mg), zirconium (Zr), zinc (Zn), and oxygen (O), respectively, before sintering. As illustrated in SEM images, the magnesium powder had an uneven flake-like shape. Moreover, the EDS mapping of elements in the mixer represented the uniform distribution of ZnO particles for Mg—ZnO composite, and similarly, the uniform distribution of Zr powder and ZnO particles was also observed for Mg—Zr—ZnO composite.

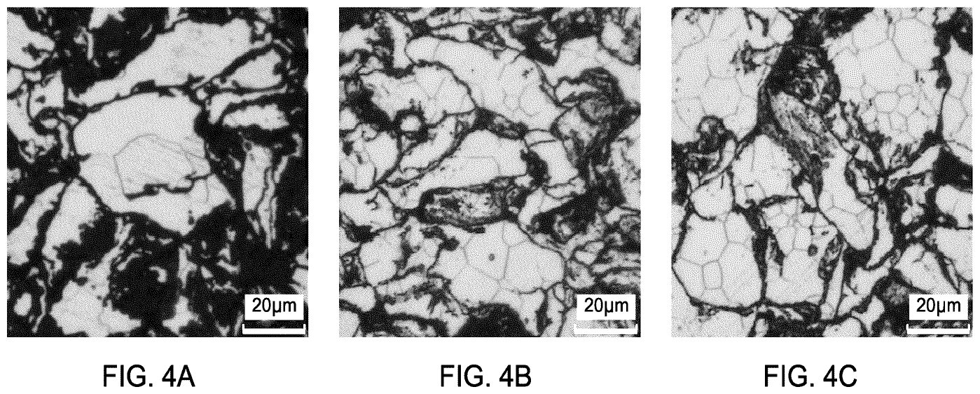

The sintered samples after etching were analyzed under a digital microscope to reveal the microstructure. FIG. 4A shows a digital micrograph of pristine Mg, FIG. 4B shows digital micrograph of Mg—ZnO composite, and FIG. 4C shows digital micrograph of Mg—Zr—ZnO composite at 20 μm scale, respectively. All samples showed irregular surface morphology due to irregular-sized Mg particles squeezed during the compaction and sintering stage. It is observed that thick and large grain boundaries in pure Mg were transformed to relatively finer grains and thin grain boundaries in Mg composites. It is also observed that the grain size in Mg composites was considerably reduced due to the presence of ZnO and Zr particles. The grain size in Mg—ZnO and Mg—Zr—ZnO composites was measured as 9.2 μm and 8.7 μm respectively as opposed to 19.5 μm for pure Mg.

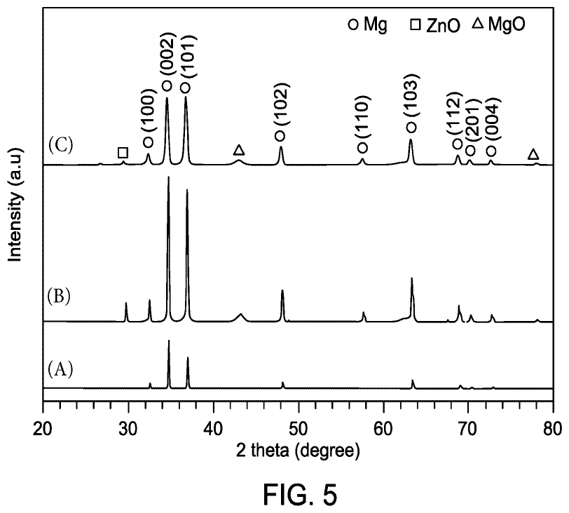

FIG. 5 shows a XRD patterns of (A) Mg, (B) Mg—ZnO composite, and (C) Mg—Zr—ZnO composite after sintering. The peaks at 2θ=32°, 35°, and 37° correspond to the prism, basal and pyramidal plane of HCP Mg-crystal respectively. No additional phases were detected in the XRD spectrum of pure magnesium from the sintering conditions utilized. The XRD analysis of pure Mg in FIG. 5A, showed the maximum intensity in the basal plane, however, the XRD peaks in Mg composites corresponding to the basal plane were lowered and were almost equal to the pyramidal plane in the case of Mg—Zr—ZnO composite. With the addition of Zr and ZnO particles, changes in the intensity of peaks can be observed which reflects the influence of reinforcing phases on the texture of magnesium crystals. In the case of the Mg—Zr—ZnO composite broadening of these peaks can be observed indicating grain refinement and a further decrease in crystallite size. In both Mg—ZnO and Mg—Zr—ZnO composites, additional peaks were observed at 20=30° and 43°, corresponding to ZnO and MgO phases respectively. This was not observed in the pristine Mg sample and thus it can be concluded that the incorporation of ZnO in Mg may result in the reaction leading to the MgO phase during sintering. It is also worth noting that no intermetallic MgZn phases were observed from the XRD pattern.

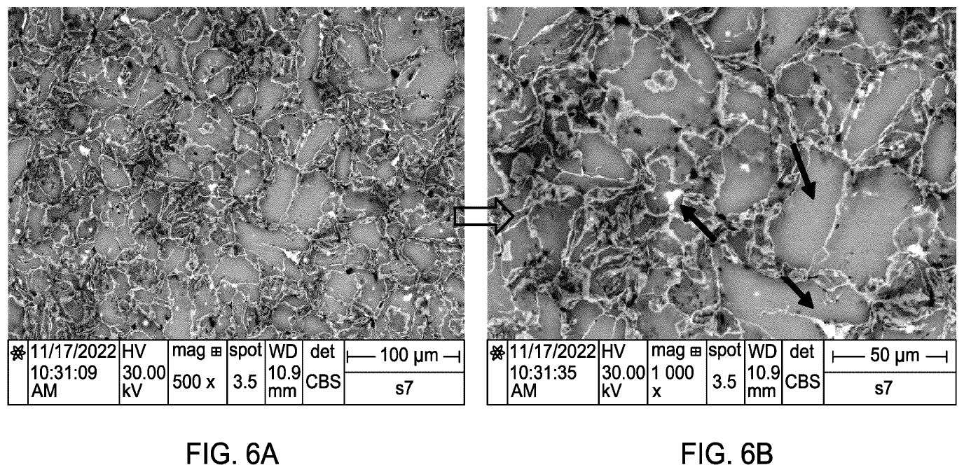

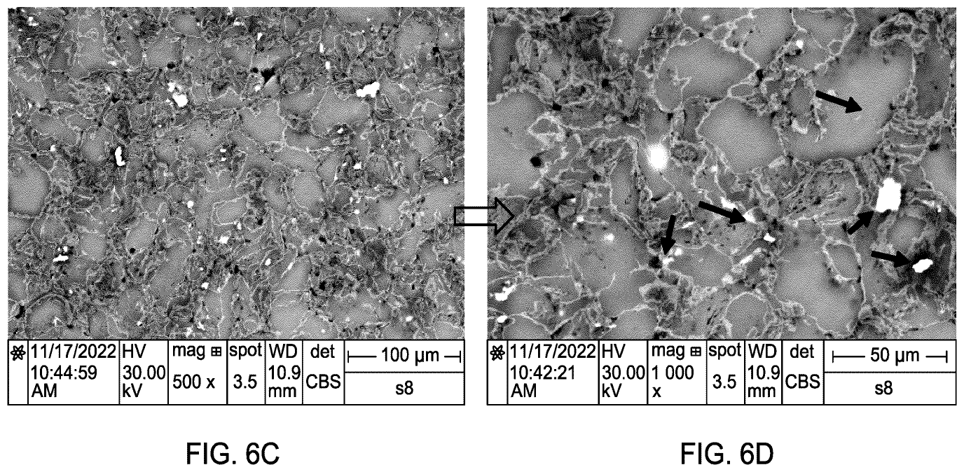

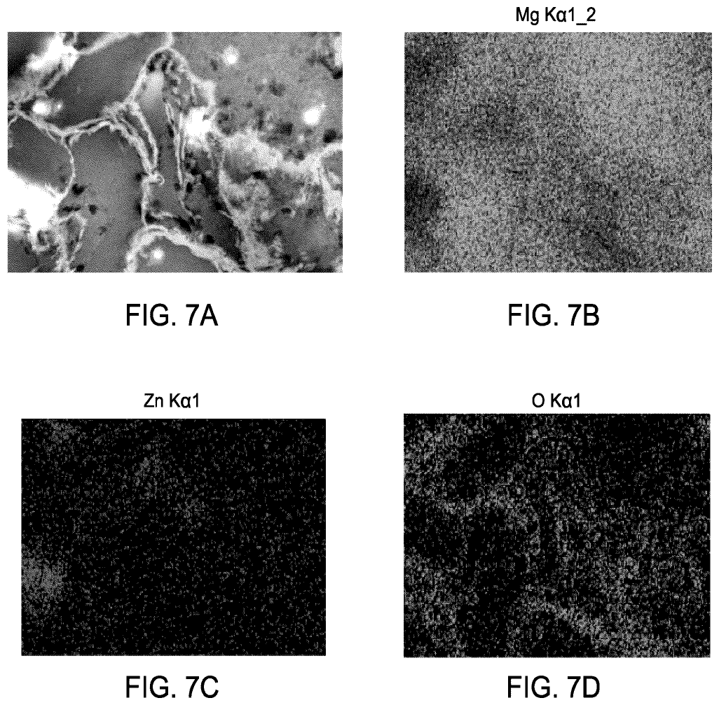

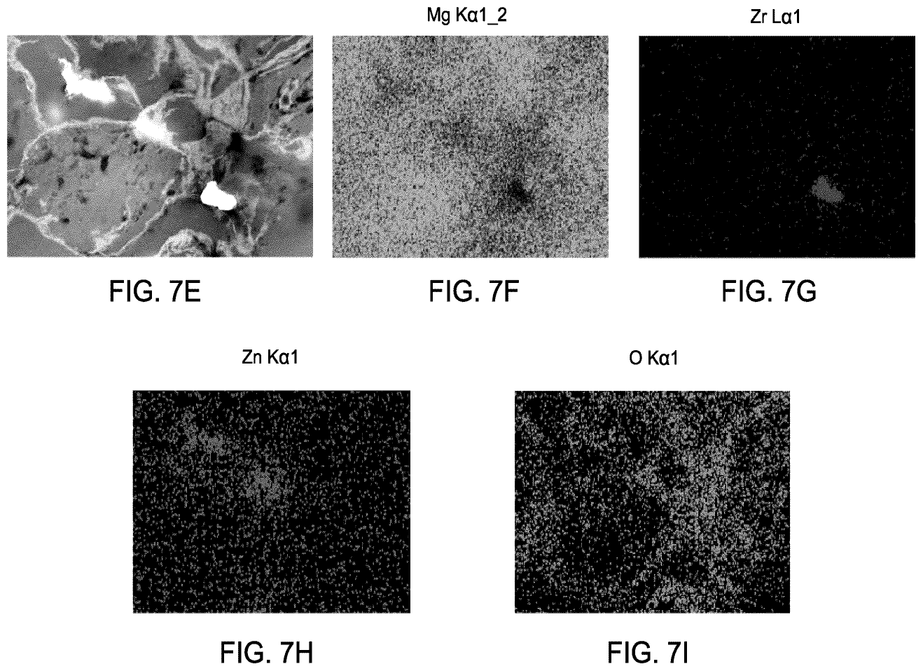

FIGS. 6A-6B show FESEM backscattered micrographs of sintered Mg—ZnO at different magnifications, while FIGS. 6C-6D show FESEM backscattered micrographs of sintered Mg—Zr—ZnO at different magnifications. Microstructural examination of the sintered Mg composite samples revealed typical random oriented α-Mg grains surrounded with boundaries and some micropores due to the effect of reinforcement particles. As observed in FIGS. 6A-6B, ZnO NPs (agglomerated) can be observed near grain boundaries with the appearance of white particles pointed in the micrograph. Relatively large and bright Zr particles were observed in the Mg—Zr—ZnO composite sample distributed in a gray Mg matrix in FIG. 6C-6D. The presence and distribution of elements are also verified by elemental mapping as shown in FIG. 7. FIG. 7A and FIG. 7E show a high magnification SEM micrograph for the sintered Mg—ZnO and Mg—Zr—ZnO composites, respectively. ZnO particles were distributed throughout the Mg matrix in both composites, however, more ZnO particles were concentrated on grain boundaries. FIGS. 7B-7D show elemental maps of the sintered Mg—ZnO composite while FIGS. 7F-7I show elemental maps of the sintered Mg—Zr—ZnO composite depicting magnesium, zinc, and oxygen, respectively. In the case of the Mg—Zr—ZnO composite, a clear distinction in elements can be observed with Zr and ZnO particles distributed in the Mg matrix and the presence of agglomeration of ZnO particles on grain boundaries some reaching close to the size of Zr in the composite sample.

The results of experimental, theoretical density, and porosity of sintered Mg, Mg—ZnO, and Mg—Zr—ZnO composite samples are shown in Table 1. The resultant average experimental density for the Mg—ZnO composite increased to 1.745 gram per cubic centimeter (g/cm³) from 1.738 g/cm³ for the only Mg composite. Whereas the density of sintered Mg—Zr—ZnO composite was measured as 1.762 g/cm³. It is worth noting that the densities of magnesium composites lie close to the densities of human bones. The average relative densities of Mg—ZnO and Mg—Zr—ZnO composites were determined to be 98.6% and 98.9% respectively. These results indicate that near-dense Mg composites were obtained using the powder metallurgy conditions utilized in this work. However, the minimal porosity of less than 2% found in composites may be due to the presence of some nanoparticle agglomeration near grain boundaries surrounding the α-Mg phase as observed in SEM micrographs (FIG. 6).

The microhardness value provides information on biomaterials' resistance to abrasion or wear in the implant's lifetime. The Vickers hardness (HV) results of sintered samples are also given in Table 1. The average hardness of the Mg—ZnO composite was measured as 73.5 showing an increment of 44% compared with pure Mg. In the case of the Mg—Zr—ZnO composite, the hardness was recorded as 76.5, showing an enhancement of 50%. This enhancement can be attributed to the grain refinement and Orowan strengthening from the synergistic effect of a homogenous dispersion of Zr and ZnO NPs in the Mg matrix.

TABLE 1
Densification and microhardness properties
of developed Mg and Mg-based composites

| | ρₜₕₑₒ | ρₑₓₚ | Porosity | Microhardness | Enhancement |
|---|---|---|---|---|---|
| Material | (g/cm³) | (g/cm³) | (%) | (HV) | % (HV) |
| Mg | 1.738 | 1.738 | — | 51 ± 3 | |
| Mg—ZnO | 1.768 | 1.745 | 1.32 ± 0.26 | 73.5 ± 4 | ≈44% |
| Mg—Zr—ZnO | 1.782 | 1.762 | 1.13 ± 0.18 | 76.5 ± 3 | ≈50% |

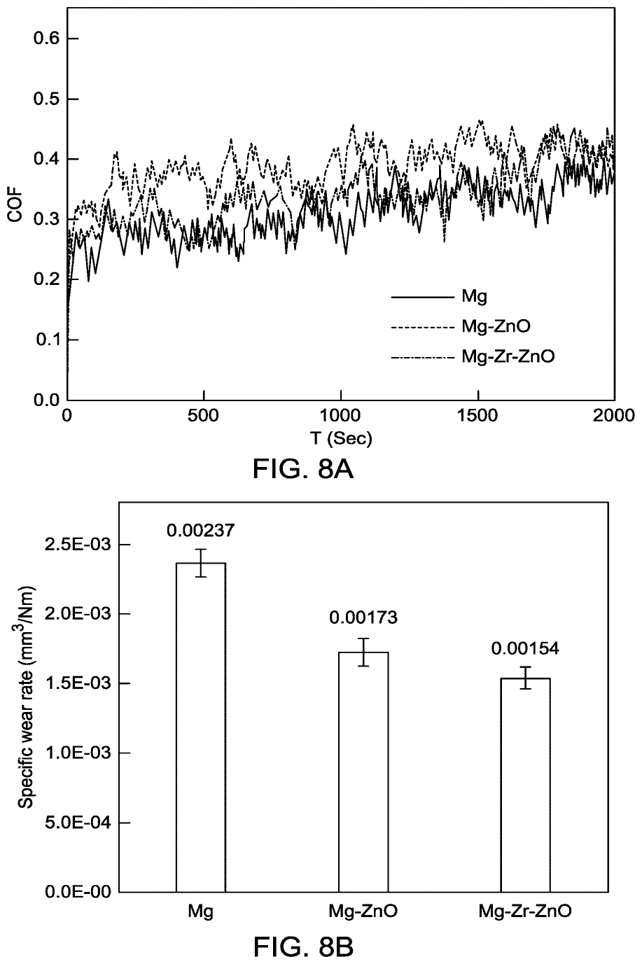

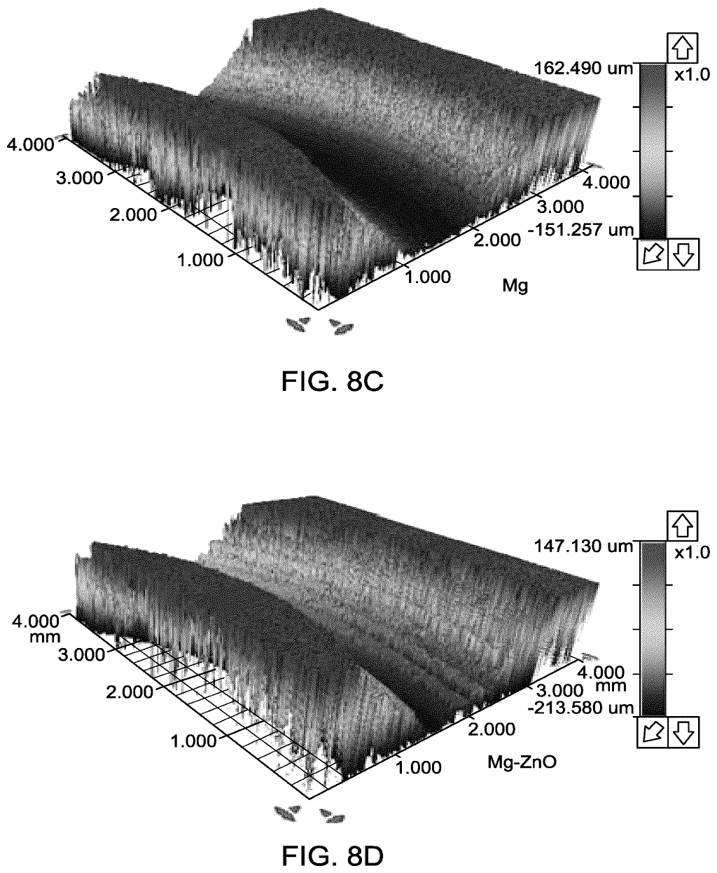

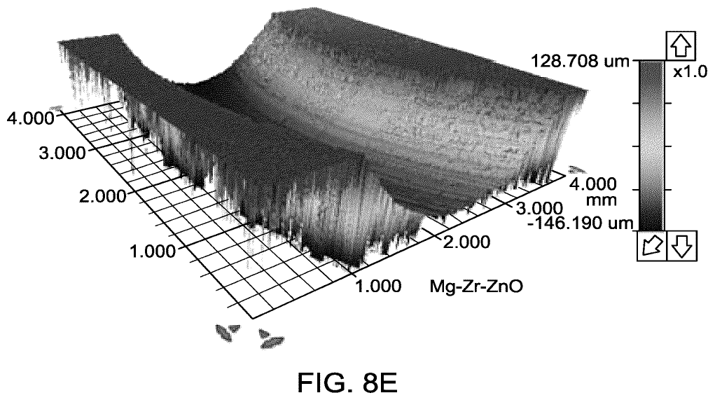

The tribological behavior of the developed Mg composites was assessed using a dry sliding wear test under suitable conditions. The coefficient of friction (COF) with respect to time during the sliding wear test is recorded in FIG. 8A. The average COF for the Mg sample was measured as 0.3 whereas for Mg—ZnO and Mg—Zr—ZnO the value increased to 0.38 and 0.35, respectively. The recorded COF values showed fluctuations and an overall increasing trend with time. This behavior was attributed to the accumulation of wear debris generated during the sliding wear test. The observed increase in COF in Mg composites was attributed to the presence of hard reinforcing particles within the Mg matrix. The ZnO and Zr particles used in this study enhanced the interfacial friction between the composite and the counterpart material. The interaction between the reinforcing particles and the counterpart material generates increased resistance to sliding, resulting in a higher COF. However, it is important to note that the observed increase in COF was not much higher. This characteristic can be advantageous in certain applications, particularly in avoiding the self-loosening of implants.

An optical profilometric study of the samples after the wear test was conducted to examine the wear track, and accurately calculate the wear rate. 3D profiles of the wear track are depicted in FIGS. 8C-8E, allowing for a visual comparison between pure Mg and Mg composites (Mg—ZnO, and Mg—Zr—ZnO composite). Notably, the wear track of pure Mg exhibited a wider and deeper profile, indicating a large wear area. In contrast, the wear tracks of Mg composites were narrow particularly the wear track of Mg—Zr—ZnO composite shows a shallow track depth representing lower wear volume. The specific wear rate measured for all samples at constant wear testing conditions is shown in FIG. 8B. The wear rate was reduced by 27% and 35% for Mg—ZnO and Mg—Zr—ZnO samples respectively. The magnesium composites developed in this study demonstrated a higher wear resistance due to the presence of Zr and ZnO particles. These particles enhance the hardness and wear resistance of the composite material by effectively distributing the applied load and creating a protective barrier against wear. This, in turn, signifies the enhanced durability and extended lifespan of the developed Mg composites in wear-intensive applications.

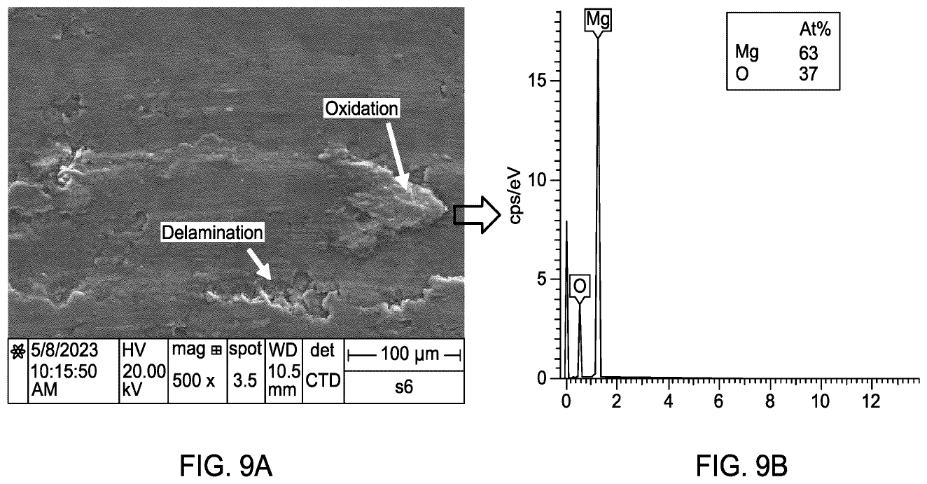

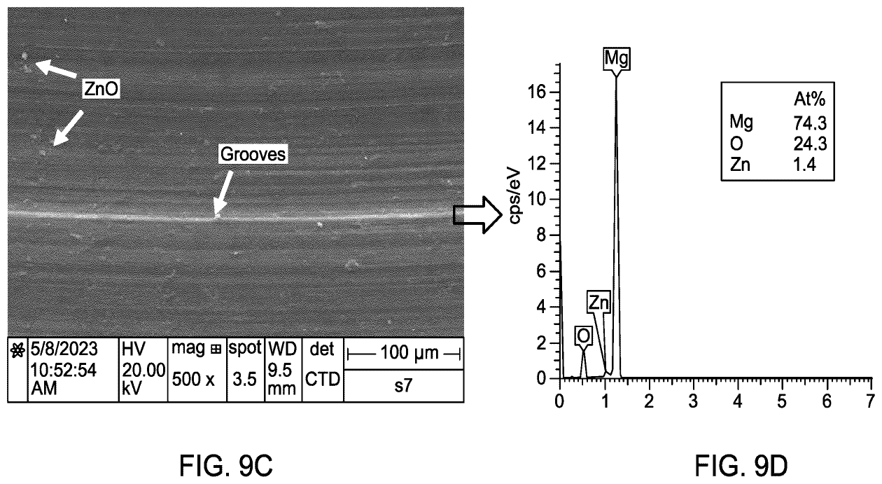

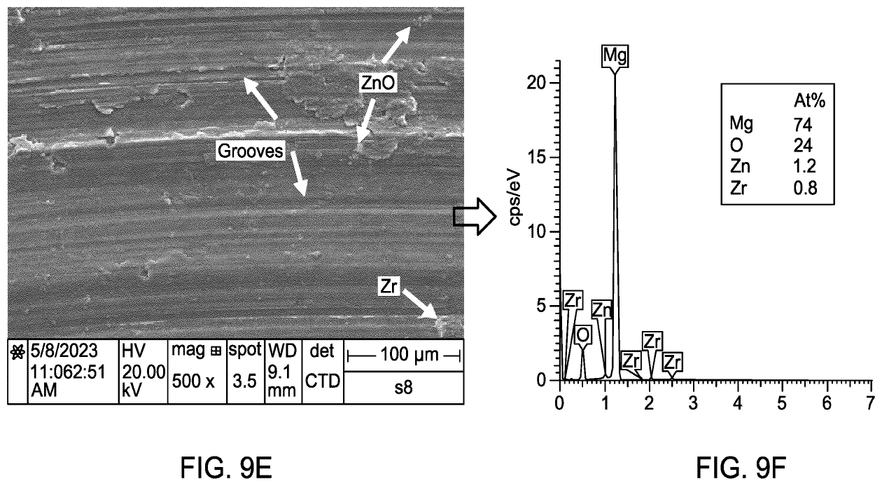

Furthermore, the structure of the worn surface was also investigated under SEM with EDS analysis to elucidate the wear mechanism and elemental composition of the material after the wear test. FIG. 9A, FIG. 9C, and FIG. 9E show the SEM image of worn surface (inside wear track core) of pure Mg, Mg—ZnO and Mg—Zr—ZnO composite, respectively. As shown in FIGS. 9C and 9E, the Mg composite samples experienced abrasive wear with the presence of shallow grooves running along the direction of sliding. These grooves were absent in pure Mg (FIG. 9A), moreover, the texture of the wear track in the Mg sample shows excessive abrasion, delamination, and surface oxidation. On the other hand, Mg—ZnO and Mg—Zr—ZnO composite worn surfaces were composed of minimum wear debris with shallow grooves in the sliding direction, attributed to the relatively hard reinforcement particles. FIG. 9B, FIG. 9D, and FIG. 9F show the corresponding EDS spectra of worn surface (inside wear track core) of pure Mg, Mg—ZnO and Mg—Zr—ZnO composites, respectively. The EDS spectrum of these composites revealed similar compositions as before the wear test indicating no external elements from the counter surface thus proving the absence of an adhesive wear mechanism. The presence of ZnO, Zr, and oxide formed during wear played a role in reducing the wear rate by minimizing direct contact between the Mg matrix and the counter surface.

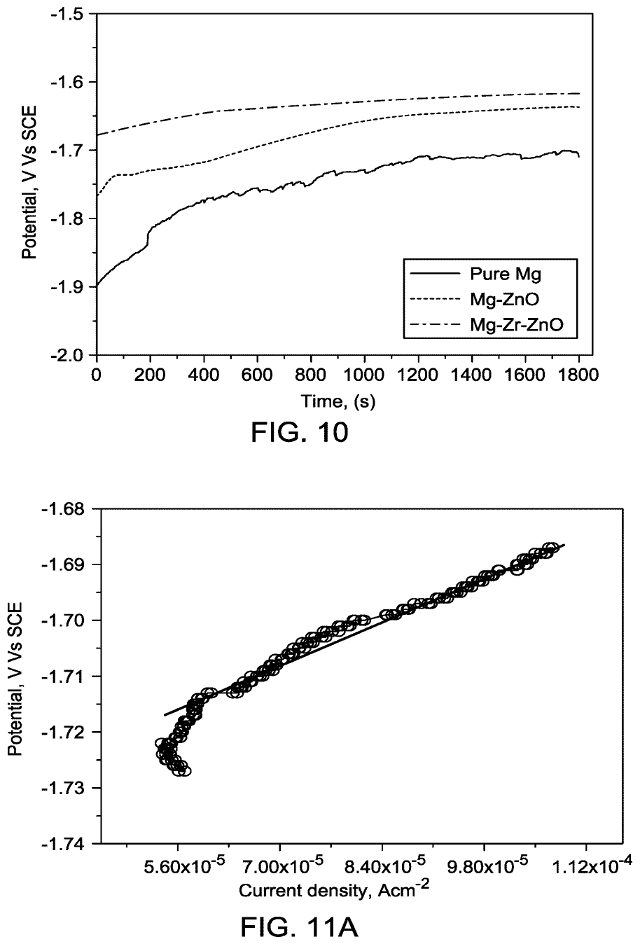

The in-vitro corrosion assessment of developed samples was done in a simulated body fluid (SBF) medium. Initial monitoring of OCP values with respect to the immersion duration was done to for preliminary results on the prepared Mg composite's corrosion resistance and FIG. 10 shows OCP curves of prepared Mg composites samples in SBF medium. For the Mg samples under investigation, the time needed to get a stable OCP value ranged from 1 min to 10 min. Compared to the examined Mg composite materials, pure Mg samples took a longer duration to attain stable OCP. The Mg—Zr—ZnO composite in particular exhibited a relatively short duration to reach a steady state with reduced variability. Additionally, the Mg—Zr—ZnO sample showed a lower corrosion potential (−1.617 V) compared to Mg—ZnO ad pure Mg samples. The Mg composite samples showed a shift in OCP when compared to the pure Mg sample, primarily showing that Mg's corrosion resistance had been improved by the addition of Zr and ZnO particles.

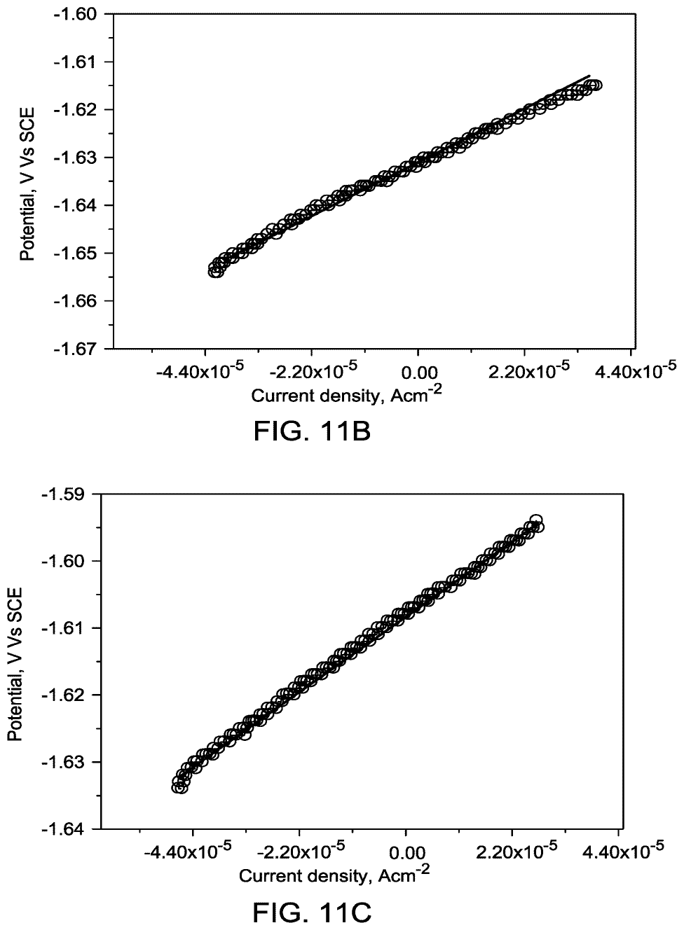

FIGS. 11A-11C present the obtained LPR curves for the prepared Mg composites and the computed values including corrosion current density (i_{corr}), corrosion potential (E_{corr}), and polarization resistance (Rₚ) are presented in Table 2. The Mg—Zr—ZnO sample demonstrated a shift in E_{corr} value over the pure Mg sample, indicating increased electrochemical stability in the SBF medium. In addition, it was identified that the i_{corr} values of pure Mg were around 92.3245 μA cm⁻², however, the value was noticeably decreased to 8.215 μA cm⁻² after adding Zr and ZnO particles to the Mg matrix, demonstrating the reduced corrosion rate of the prepared Mg composite. Furthermore, the prepared Mg—Zr—ZnO sample had a higher Rₚ value than the pure Mg sample, which further supports the impression that adding Zr and ZnO particles improved the corrosion-resistant behavior.

TABLE 2
LPR values of prepared Mg composites in SBF medium.

| | | | | | Rₚ |
|---|---|---|---|---|---|
| Substrate | E_{corr}^{V} | I_{corr}^{μA cm}^{^{−2}} | βₐ^{mV/dec} | β_{b}^{mV/dec} | Ω cm² |
| Pure Mg | −1.6945 | 92.3245 | 67 | 92 | 182.25 |
| Mg—ZnO | −1.637 | 22.309 | 73 | 75 | 719.59 |
| Mg—Zr—ZnO | −1.617 | 8.215 | 80 | 69 | 1957.64 |

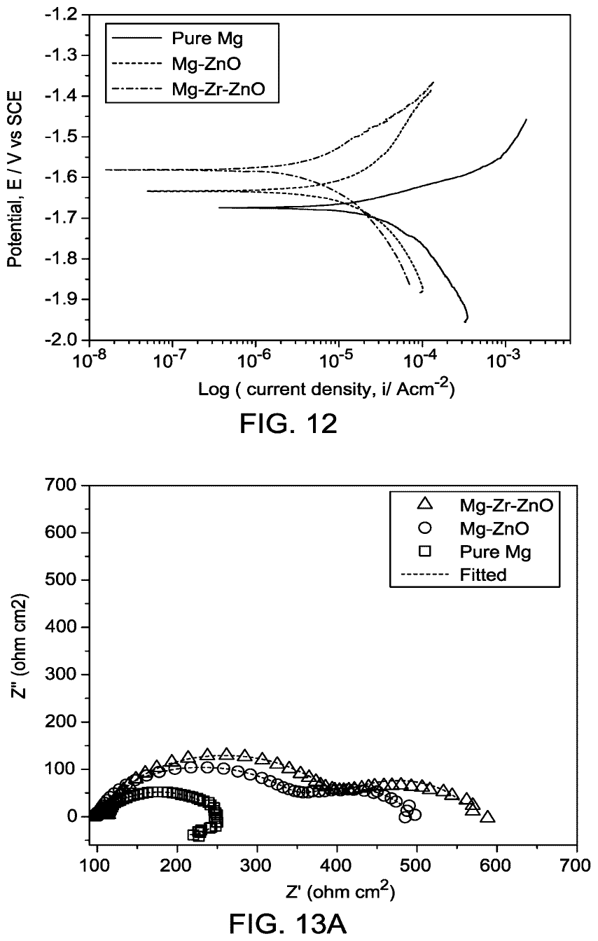

PDP curves for the prepared Mg composite samples in SBF medium are presented in FIG. 12 and the extracted values from Tafel analysis are given in Table 3. Mg shows an electrochemically higher activity without exhibiting passivation characteristics and also the cathodic and anodic branches of PDP of Mg represent the hydrogen evolution and Mg dissolution, respectively. Pure Mg sample exhibited the higher i_{corr} and most negative E_{corr}, validating its inadequate corrosion-resistant performance in SBF.

The prepared Mg composite showed a lower i_{corr} and higher E_{corr}, confirming the improved corrosion-resistant action due to Zr and ZnO particles. Table 3 shows that the estimated corrosion rate of the Mg composite is lower than that of pure Mg, further supporting the favorable synergistic impact of the Zr and ZnO particles' reinforcement in the Mg matrix on the anti-corrosion behavior in the SBF medium.

TABLE 3
Tafel parameters of prepared Mg composites in SBF medium

| | | | | | Corr. Rate |
|---|---|---|---|---|---|
| Substrate | E_{corr}^{V} | I_{corr}^{μA cm}^{^{−2}} | βₐ^{mV/dec} | β_{b}^{mV/dec} | (mm/yr) |
| Pure Mg | −1.6921 | 124.52 | 89 | 73 | 2.845 |
| Mg—ZnO | −1.631 | 7.369 | 63 | 73 | 0.168 |
| Mg—Zr—ZnO | −1.597 | 2.456 | 85 | 94 | 0.056 |

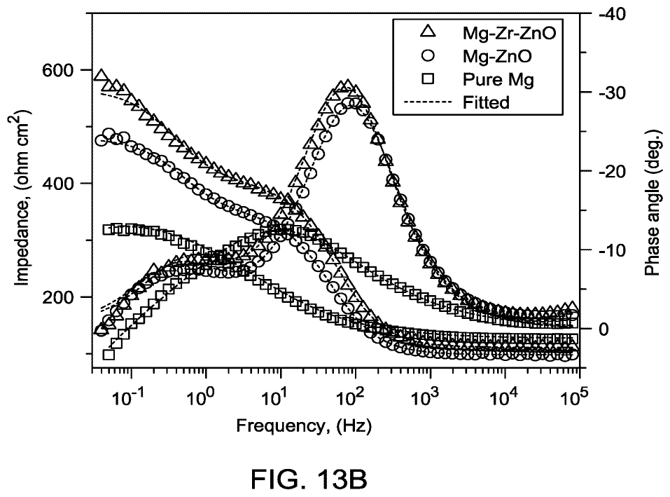

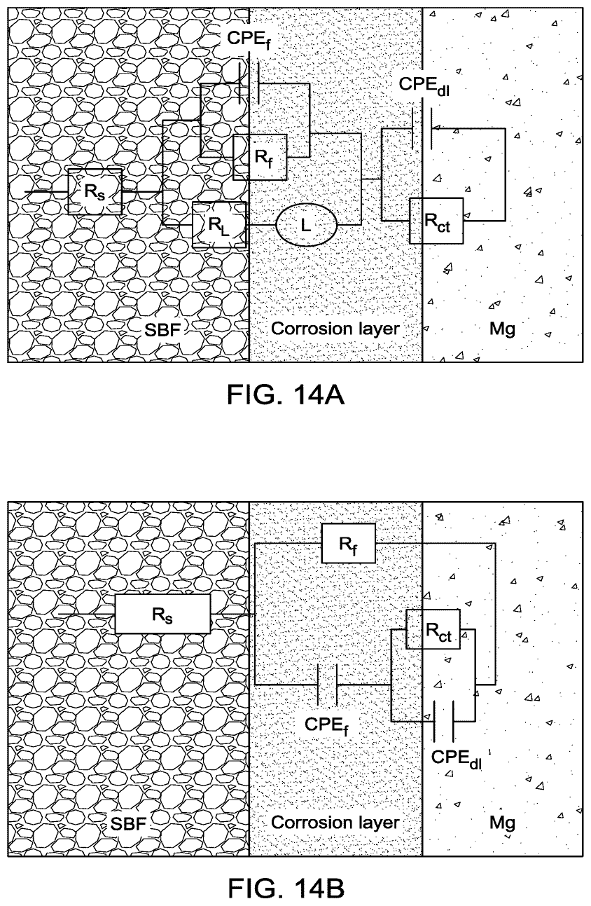

FIG. 13A shows the Nyquist curves of the investigated Mg samples in the SBF medium. At the high and intermediate frequencies, one distorted capacitive arc was seen, followed by an inductive loop at the low-frequency zone. The electrochemical double layer and charge transfer response are responsible for the capacitive arc in high-frequency regions, while the surface-attached Mg intermediates, such as Mg⁺ and Mg (OH)⁺ ions, are responsible for the inductive loop at low frequencies. Bode plots of Mg composites are shown in FIG. 13B. At the lowest measured frequency, the pure Mg exhibited the lowest impedance value of 321 Ω cm², while it was found to be about 588 Ω cm² for Mg—Zr—ZnO composite sample.

In general, the higher impedance modulus at the lowest frequency is an obvious sign of the higher corrosion-resistant performance of the analyzed material, and therefore, the surface protective performance provided by the analyzed materials can be graded as pure Mg<Mg-ZnO<Mg-Zr—ZnO. FIG. 14A and FIG. 14B show EIS circuit model for pure Mg and Mg composites in SBF medium. By selecting the proper EIS model (FIG. 14A-14B), an equivalent circuit fitting analysis was carried out to further quantitatively examine the acquired EIS curves, and the obtained values are summarized in Table 4.

TABLE 4
EIS parameters of prepared Mg composites in SBF medium

| | Rₛ | R_{ct} | CPE_{dl} | |
|---|---|---|---|---|
| Substrate | Ω cm² | Ω cm² | (Ω⁻¹ cm⁻²sⁿ) | n_{dl} |
| Pure Mg | 99.45 | 321.55 | 129.87 | 0.94 |
| Mg—ZnO | 102.34 | 487.93 | 43.256 | 0.96 |
| Mg—Zr—ZnO | 100.25 | 588.92 | 23.457 | 0.97 |

By comparing the acquired Ret values, the Mg—Zr—ZnO composite had the highest Ret value, proving its improved corrosion-resistant action in the SBF medium. Additionally, the CPE_{dl} value of the Mg composite is revealed to be one order of magnitude lower than that of pure Mg, confirming that the reinforcement of Zr and ZnO particles in the Mg matrix prevents the permeation of aggressive ions from the SBF towards the Mg surface.

The microhardness, wear rate, and in vitro corrosion study of Mg-based composites developed in the current work were compared with similar investigations reported in the literature. As shown in Table 5, few studies have reported mechanical, wear, and corrosion properties all together for Mg-based nanocomposites for biomedical applications. The developed composited showed enhanced microhardness and corrosion resistance due to the inclusion of ZnO in nanoparticle and refined microstructure. The enhanced microhardness, wear, and in vitro corrosion properties of the processed composited make it a potential for bioimplant applications.

TABLE 5
Microhardness, wear, and in vitro corrosion properties of the processed
Mg-based nanocomposites in comparison to previous studies

| Material | Microhardness | Wear rate | Corrosion rate | |
|---|---|---|---|---|
| (% in wt.) | (HV) | (mm³/m) | mm/yr. | Reference |
| Mg—2.5% ZnO | 73.5 | 1.72 × 10⁻⁴ | 0.168 (SBF) | This work |
| Mg—1% Zr—2.5% ZnO | 76.5 | 1.53 × 10⁻⁴ | 0.056 (SBF) | This work |
| Mg—1% Zr | 56.6 | — | 3.032. (Hanks) | [30] |
| Mg—20% ZnO | 66.2 | — | 1.10 (SBF) | [39] |
| Mg—2.5% TiB₂ | 53.77 | — | 0.2647 (SBF) | [15] |
| Mg—1.5Zn—0.5 | 53.5 | 3.6 × 10⁻³ | | [52] |
| Mn—0.6Si + 2% HA | | | | |
| AZ31 + 3% SiO₂ | 73 | — | 0.3 (PBS) | [53] |
| Mg—12.5% HA—10% | — | — | 1.06 (SBF) | [54] |
| MgO | | | | |
| AZ91D + 3% TiC | 55.7 | | 0.1165 (SBF) | [55] |
| AZ91 + 20% FA | 93 | — | 26.68 (SBF) | [56] |
| AZ91 + 10% TiB₂ | 102 | 0.011 | — | [57] |

[30]M. A. Hussein, M. A. Azeem, A. M. Kumar, N. M. Emara, Processing and in vitro corrosion analysis of sustainable and economical eggshell reinforced Mg and Mg—Zr matrix composite for biomedical applications, Mater. Today Commun. 32 (2022).
[39]T. Lei, W. Tang, S.-H. Cai, F.-F. Feng, N.-F. Li, On the corrosion behaviour of newly developed biodegradable Mg-based metal matrix composites produced by in situ reaction, Corros. Sci. 54 (2012) 270-277.
[15]M. A. Hussein, M. A. Azeem, A. Madhan Kumar, M. Ali, A. Alghanim, Processing, Characterization, and In Vitro Corrosion Behavior of Mg—TiB2 Composite for Orthopedic Applications, JOM. 74 (2022) 981-989.
[52]P. S. Kumar, K. Ponappa, M. Udhayasankar, B. Aravindkumar, Dry Sliding Wear and Mechanical Characterization of Mg Based Composites by Uniaxial Cold Press Technique, Arch. Metall. Mater. 62 (2017).
[53]A. K. M. A. Iqbal, N. B. Ismail, Mechanical Properties and Corrosion Behavior of Silica Nanoparticle Reinforced Magnesium Nanocomposite for Bio-Implant Application, Materials (Basel). 15 (2022).
[54]S. Z. Khalajabadi, M. R. Abdul Kadir, S. Izman, R. Ebrahimi-Kahrizsangi, Fabrication, bio-corrosion behavior and mechanical properties of a Mg/HA/MgO nanocomposite for biomedical applications, Mater. Des. 88 (2015) 1223-1233.
[55]G. Anbuchezhiyan, B. Mohan, S. Kathiresan, R. Pugazenthi, Influence of microstructure and mechanical properties of TiC reinforced magnesium nano composites, Mater. Today Proc. 27 (2020) 153β-1534.
[56]M. Razavi, M. H. Fathi, M. Meratian, Bio-corrosion behavior of magnesium-fluorapatite nanocomposite for biomedical applications, Mater. Lett. 64 (2010) 2487-2490.
[57]F. Aydin, Y. Sun, M. Emre Turan, The Effect of TiB₂ Content on Wear and Mechanical Behavior of AZ91 Magnesium Matrix Composites Produced by Powder Metallurgy, Powder Metall. Met. Ceram. 57 (2019) 564-572.

Numerous modifications and variations of the present disclosure are possible in light of the above teachings. It is therefore to be understood that within the scope of the appended claims, the invention may be practiced otherwise than as specifically described herein.

1: A method of making a composite, comprising:
mixing ZnO nanoparticles (NPs), Mg particles, and Zr particles under an inert atmosphere to form a powder mixture;
compacting the powder mixture at a pressure of 500-600 MPa for at least 1 minute to form a compacted mixture; and
sintering the compacted mixture at a temperature of 400-500° C. for at least 1 hour to form the composite,
wherein the composite includes 1-10 wt. % of the ZnO NPs and 0.1-5 wt. % of the Zr particles, based on a total weight of the composite,
wherein the Zr particles and the ZnO NPs are homogeneously dispersed in a matrix of the Mg particles in the composite,
wherein the Mg particles have an average grain size of 5-10 μm in the composite, and
wherein the Zr particles and the ZnO NPs separately form aggregates at grain boundaries of the Mg particles in the composite.

2: The method of claim 1, wherein in the mixing the ZnO NPs have an average size of 80-200 nm.

3: The method of claim 1, wherein in the mixing the Mg particles have an average particle size of 30-60 μm.

4: The method of claim 1, wherein in the mixing the Zr particles have an average particle size of 30-60 μm.

5: The method of claim 1, wherein the composite includes Mg, ZnO, and MgO crystal phases.

6: The method of claim 1, wherein the composite does not include a MgZn crystal phase.

7: The method of claim 1, wherein the Mg particles include α-Mg in the composite.

8: The method of claim 1, wherein the aggregates of the ZnO NPs are from 0.5-10 μm in size.

9: The method of claim 1, wherein the aggregates of the Zr particles are from 0.5-10 μm in size.

10: The method of claim 1, wherein the composite has a density of 1.7-1.8 g/cm³.

11: The method of claim 1, wherein the composite has less than 2% porosity.

12: The method of claim 1, wherein the composite has a Vickers hardness of 70-80 HV.

13: The method of claim 1, wherein the composite has a specific wear rate of 0.001 to 0.002 mm³/Nm.

14: The method of claim 1, wherein the composite has a corrosion rate of less than 0.1 mm/yr in a human body fluid solution.

15: The method of claim 1, wherein the mixing homogeneously disperses the Zr particles and the ZnO NPs in the Mg particles.

16: The method of claim 1, wherein the sintering produces Mg, ZnO, and MgO crystal phases.

17: A composite made by the method of claim 1.

18: An implant comprising the composite of claim 17.

19: The implant of claim 18, having a corrosion rate of less than 0.1 mm/yr in a human body fluid solution.
